(19) United States
(12) Patent Application Publication
(10) Pub. No.: US 2014/0024609 A1
(43) Pub. Date: Jan. 23, 2014

(54) Treatment of Bacterial Infections

(71) Applicants: Yoshi Ichikawa, San Diego, CA (US); Sherwood Gorbach, San Diego, CA (US)

(72) Inventors: Yoshi Ichikawa, San Diego, CA (US); Sherwood Gorbach, San Diego, CA (US)

(73) Assignee: Optimer Pharmaceuticals ,Inc., Jersey City, NJ (US)

(21) Appl. No.: 13/983,440

(22) PCT Filed: Feb. 6, 2012
(86) PCT No.: PCT/US2012/024005

§ 371 Date: Oct. 3, 2013

Related U.S. Application Data
(60) Provisional application No. 61/439,673, filed on Feb. 4, 2011.
(60) Provisional application No. 61/552,383, filed on Oct. 27, 2011.

(51) Int. Cl. A61K 31/7048; A61K 31/58; A61K 31/606

(52) U.S. Cl. CPC A61K 31/7048; A61K 31/606; A61K 31/58
USPC 514/28

(57) ABSTRACT

The present invention relates to methods of treating a bacterial infection in a subject in need thereof comprising administering to the subject an effective amount of the compound of Formula (I) and an anti-inflammatory agent. The present invention provides pharmaceutical compositions comprising an effective amount of the compound of Formula (I) and an anti-inflammatory agent.

### FIELD OF THE INVENTION

The present invention relates to the use of an effective amount of a compound of Formula I and an anti-inflammatory agent to treat a bacterial infection in a subject in need thereof. The present invention also relates to pharmaceutical compositions comprising an effective amount of the combination of compound of Formula I and an anti-inflammatory agent.

### BACKGROUND OF THE INVENTION

Many antibiotics alter the balance among the types and quantity of bacteria in the intestine, thus allowing certain disease-causing bacteria to multiply and replace other bacteria. Such bacteria include Clostridium spp., Staphylococcus spp. and Enterococcus spp. One particular bacterium that commonly results in infection is Clostridium difficile (C. difficile). The colonized C. difficile releases two toxins (toxin A and toxin B) that can cause inflammation of the protective lining of the large intestine (colitis) leading to diarrhea, which results in C. difficile infections (also referred to as C. difficile-associated diarrhea or C. difficile-induced colitis). C. difficile is responsible for approximately 20% of the cases of antibiotic-associated diarrhea (AAD) and the majority of the cases of antibiotic-associated colitis (AAC).

There are currently two dominant therapies for C. difficile-associated diarrhea (CDAD): vancomycin and metronidazole. Vancomycin is not recommended for first-line treatment of CDAD mainly because it is the only antibiotic active against several serious life-threatening multi-drug resistant bacteria. Therefore, in an effort to minimize the emergence of vancomycin-resistant Enterococcus (VRE) or vancomycin-resistant S. aureus (VRSA), the medical community discourages the use of vancomycin except when absolutely necessary.

Metronidazole is recommended as initial therapy out of concern for the promotion and selection of vancomycin resistant gut flora, especially enterococci. Despite reports that the frequency of C. difficile resistance may be >6% in some countries, metronidazole remains nearly as effective as vancomycin, is considerably less expensive, and can be used either orally or intravenously. Metronidazole is associated with significant adverse effects including nausea, neuropathy, leukopenia, seizures, and a toxic reaction to alcohol. Furthermore, it is not safe for use in children or pregnant women.

Tiacumicins, specifically Tiacumicin B, show activity against a variety of bacterial pathogens and in particular against C. difficile (Antimicrob. Agents Chemother. 1991, 1108-1111). Tiacumicin B shows promising activity against C. difficile, and is expected to be useful in the treatment of bacterial infections, especially those of the gastrointestinal tract, in mammals. Examples of such treatments include, but are not limited to, treatment of colitis and treatment of irritable bowel syndrome.

Tiacumicin antibiotics are described in U.S. Pat. No. 4,918,174 (issued Apr. 17, 1990), J. Antibiotics 1987, 40: 575-588, J. Antibiotics 1987, 40: 567-574, J. Liquid Chromatography 1988, 11: 191-201, Antimicrobial Agents and Chemotherapy 1991, 35: 1108-1111, U.S. Pat. No. 5,583,115 (issued Dec. 10, 1996), and U.S. Pat. No. 5,767,096 (issued Jun. 16, 1998), which are all incorporated herein by reference. Related compounds are the Lipiarmycin antibiotics (c.f., J. Chem. Soc. Perkin Trans. I, 1987, 1353-1359 and J. Antibiotics 1988, 41: 308-315) and the Clostomicin antibiotics (J. Antibiotics 1986, 39: 1407-1412), which are all incorporated herein by reference.

C. difficile is a leading cause of hospital acquired diarrhea, which exhibits a high mortality among elderly and immunocompromised patients. It is therefore desirable for any therapy directed to treating these antibiotic-associated infections to exhibit the following two properties: antibacterial activity against the bacterium (e.g., C. difficile) and anti-inflammatory activity to shorten the symptoms and reduce the fluid loss resulting from the diarrhea.

### SUMMARY OF THE INVENTION

The present invention relates to a method of treating bacterial infections in a subject in need thereof comprising administering to the subject an effective amount of a compound of Formula I and an anti-inflammatory agent.

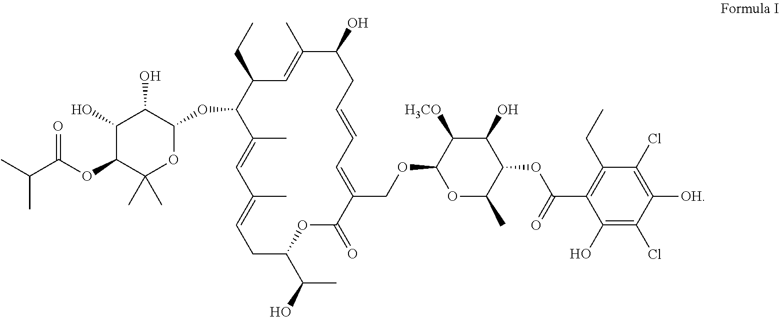 [SMILES: CCc1c(Cl)c(O)c(Cl)c(O)c1C(=O)O[C@H]1[C@H](O)[C@H](OC)[C@H](OC/C2=C\C=C\C[C@H](O)/C(C)=C/[C@H](CC)[C@@H](O[C@@H]3OC(C)(C)[C@@H](OC(=O)C(C)C)[C@H](O)[C@@H]3O)/C(C)=C/C(C)=C/C[C@@H]([C@@H](C)O)OC2=O)O[C@@H]1C]

In an exemplary embodiment, the infection is due to a gram-positive bacterium.

In an exemplary embodiment, the infection is due to a gram-negative bacterium.

In an exemplary embodiment, the infection is due to Clostridium spp., Staphylococcus spp., Streptococcus spp., Enterococcus spp. or combinations thereof.

In an exemplary embodiment, the Staphylococcus spp. is methicillin-resistant Staphylococcus spp.

In an exemplary embodiment, the infection is due to Clostridium spp.

In an exemplary embodiment, the infection is due to Clostridium difficile, Clostridium perfringens or Staphylococcus aureus.

In an exemplary embodiment, the infection appears as a disease or disorder selected from the group consisting of C. difficile-associated diarrhea, C. difficile-associated colitis and inflammatory bowel disease.

In an exemplary embodiment, the compound of Formula I is administered as a pharmaceutical composition.

In an exemplary embodiment, the pharmaceutical composition of Formula I further comprises butylated hydroxy toluene.

In an exemplary embodiment, the pharmaceutical composition of Formula I is administered orally.

In an exemplary embodiment, the therapy for treatment of inflammation comprises administration of an anti-inflammatory agent by an intramuscular, intraperitoneal, intranasal, oral, sublingual, intravaginal or rectal route.

In an exemplary embodiment, the anti-inflammatory agent is delivered rectally.

In an exemplary embodiment, the combination of the compound of Formula I with an anti-inflammatory agent is administered as a solid dosage formulation.

In an exemplary embodiment, the anti-inflammatory agent is mesalamine (5-aminosalicylic acid (5-ASA), Asacol®, Pentasa®, Rowasa), sulfasalazine (Azulfidine®), balsalazide (Colazal®), inflixamab (Remicade®), olsalazine (Dipentum®), or budesonide (Entocort EC®).

In an exemplary embodiment, the anti-inflammatory is administered as a pharmaceutical composition comprising an excipient.

In an exemplary embodiment, the compound of Formula I contains at least 93% of the R-stereoisomer.

In an exemplary embodiment, the subject is a mammal. In a particular embodiment, the mammal is a human.

The present invention also relates to pharmaceutical compositions comprising an effective amount of a combination of a compound of Formula I

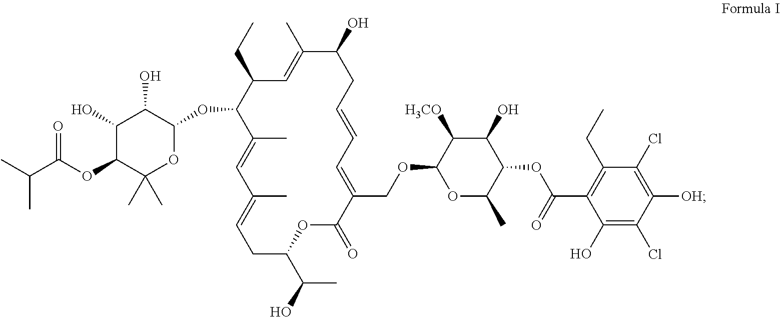 [SMILES: CCc1c(Cl)c(O)c(Cl)c(O)c1C(=O)O[C@H]1[C@H](O)[C@H](OC)[C@H](OC/C2=C\C=C\C[C@H](O)/C(C)=C/[C@H](CC)[C@@H](O[C@@H]3OC(C)(C)[C@@H](OC(=O)C(C)C)[C@H](O)[C@@H]3O)/C(C)=C/C(C)=C/C[C@@H]([C@@H](C)O)OC2=O)O[C@@H]1C]

and an anti-inflammatory agent.

In an exemplary embodiment, the anti-inflammatory agent is selected from the group consisting of mesalamine, sulfasalazine, balsalazide, inflixamab, olsalazine, and budesonide.

In an exemplary embodiment, the anti-inflammatory agent is mesalamine.

In an exemplary embodiment, the anti-inflammatory agent is budesonide.

In an exemplary embodiment, the anti-inflammatory agent is a 5-aminosalicylic acid compound.

### BRIEF DESCRIPTION OF DRAWINGS

FIG. 1 shows the X-ray power diffraction patterns of a first polymorph of the compound of Formula I produced from methanol and water.

FIG. 2 shows the X-ray power diffraction patterns of a second polymorph of the compound of Formula II produced from ethyl acetate.

FIG. 3 shows the Kaplan-Meier survival plots for mice with different doses of C. difficile VP11063 (2×10², 10³, 10⁴, 10⁵ cfu) after 3 days of antibiotic pretreatment and a single dose of clindamycin (n=12 per group except n=24 for the 10⁵ group).

FIG. 4 shows the histologic features of colonic tissues obtained from antibiotic-treated mice exposed to C. difficile.

### DETAILED DESCRIPTION OF THE INVENTION

The present invention relates to a method of treating a bacterial infection in a mammal comprising administering to the mammal an amount of a compound of Formula I

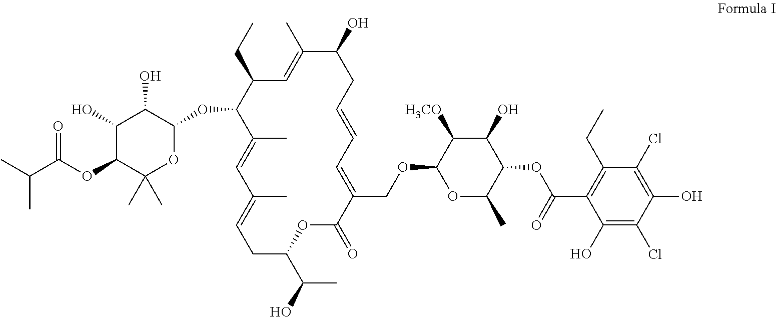 [SMILES: CCc1c(Cl)c(O)c(Cl)c(O)c1C(=O)O[C@H]1[C@H](O)[C@H](OC)[C@H](OC/C2=C\C=C\C[C@H](O)/C(C)=C/[C@H](CC)[C@@H](O[C@@H]3OC(C)(C)[C@@H](OC(=O)C(C)C)[C@H](O)[C@@H]3O)/C(C)=C/C(C)=C/C[C@@H]([C@@H](C)O)OC2=O)O[C@@H]1C]

and an anti-inflammatory agent.

The present invention also relates to pharmaceutical compositions comprising an effective amount of a combination of a compound of Formula I

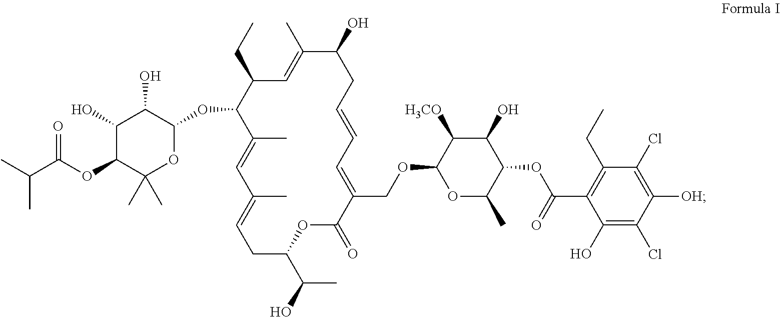 [SMILES: CCc1c(Cl)c(O)c(Cl)c(O)c1C(=O)O[C@H]1[C@H](O)[C@H](OC)[C@H](OC/C2=C\C=C\C[C@H](O)/C(C)=C/[C@H](CC)[C@@H](O[C@@H]3OC(C)(C)[C@@H](OC(=O)C(C)C)[C@H](O)[C@@H]3O)/C(C)=C/C(C)=C/C[C@@H]([C@@H](C)O)OC2=O)O[C@@H]1C]

and an anti-inflammatory agent.

As used herein, “fidaxomicin” refers to the therapeutically active composition tested in the Examples described herein that comprises the compound of Formula I. HPLC analysis showed fidaxomicin to contain about >93% of compound of Formula I as a major component and a mixture of tiacumicins as the minor component.

As used herein, the term “treatment” indicates a procedure which is designed ameliorate one or more causes, symptoms, or untoward effects of a bacterial infection in a subject. Likewise, the term “treat” is used to indicate performing a treatment. The treatment can, but need not, cure the subject, i.e., remove the cause(s), or remove entirely the symptom(s) and/or untoward effect(s) of the bacterial infection in the subject. Thus, a treatment may include treating a subject to inhibit the growth or proliferation of bacteria or protozoa, e.g., C. difficile, in the subject, or it may attenuate symptoms such as, but not limited to, diarrhea, fever, cramps, dehydration and peritonitis, or may include removing or decreasing the severity of the root cause of the bacterial infection in the subject. Treatment of a bacterial infection also includes treating after-arising symptoms that are related to the initial infection, such as diarrhea, fever, cramps, dehydration and peritonitis.

As used herein, the term “subject” is used interchangeably with the term “patient,” and is used to mean an animal, in particular a mammal, and even more particularly a non-human or human primate.

As used herein, the term “5-aminosalicylic acid (5-ASA) compound” refers to a compound which incorporates all or a part of the structure of 5-ASA while still retaining the anti-inflammatory effects of 5-ASA.

A “bacterial infection” is used herein as it is used in the art, and the phrase is also used herein to include protozoal infections as well as disorders, conditions or symptoms associated with the bacterial infection and/or protozoal infections. In one embodiment, the bacterial infection is an infection of Clostridium difficile (C. difficile), Staphylococcus spp., including but not limited to methicillin-resistant S. aureus (MRSA), Enterococcus spp. including but not limited to vancomycin-resistant Enterococci (VRE) or Clostridium perfringens (C. perfringens). The bacterial infection can be in any system, organ, tissue or area of the subject, such as but not limited to, gastrointestinal including upper and/or lower portions thereof, urinary, skin, ocular, auditory, blood, and respiratory to name a few.

Other bacterial infections and disorders related to such infections include but are not limited to disorders associated with the use of antibiotics, chemotherapies, or antiviral therapies, including, but not limited to, colitis, for example, pseudo-membranous colitis, antibiotic associated diarrhea. More specifically, antibiotic-associated diarrhea caused by toxin producing strains of C. difficile, S. aureus including methicillin-resistant S. aureus, and C. perfringens. Others include antibiotic-associated colitis, pneumonia, otitis media, sinusitis, bronchitis, tonsillitis and mastoiditis related to infection by S. pneumoniae, Haemophilus influenzae, Moraxella catarrhalis, S. aureus, or Peptostreptococcus spp., pharynigis, rheumatic fever and glomerulonephritis related to infection by S. pyogenes, Groups C and G streptococci, C. diptheriae or Actinobacillus haemolyticum. Still others include respiratory tract infections related to infection by Mycoplasma pneumoniae, Legionella pneumophila, Streptococcus pneumoniae, Haemophilus influenzae, or Chlamydia pneumoniae, uncomplicated skin and soft tissue infections, abscesses and osteomyelitis, and puerperal fever related to infection by S. aureus, coagulase-positive Staphylococci (e.g., S. epidermis and S. hemolyticus), S. pyogenes, S. agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Neiserria gonorrhea. Other include toxin diseases related to infection by S. aureus (food poisoning and Toxic Shock Syndrome), or Groups A, B and C streptococci; ulcers related to infection by Helicobacter pylori, systemic febrile syndromes related to infection by Borrelia recurrentis; Lyme disease related to infection by Borrelia burgdorferi, conjunctivitis, keratitis, and dacrocystitis related to infection by Chlamydia trachomatis, Neisseria gonorrhoeae, S. aureus, S. pneumoniae, S. pyogenes, H. influenzae, or Listeria spp. Others include disseminated Mycobacterium avium complex (MAC) diseases related to infection by Mycobacterium avium, or Mycobacterium intracellular; gastroenteritis related to infection by Campylobacter jejuni, intestinal protozoa related to infection by Cryptosporidium spp., odontogenic infection related to infection by viridans streptococci; persistent cough related to infection by Bordetella pertussis, gas gangrene related to infection by C. perfringens or Bacteroides spp., and atherosclerosis related to infection by H. pylori or Chlamydia pneumoniae. Other bacterial infections that may be treated, prevented or the likelihood of occurrence of which may be reduced in accord with the methods of the invention are referred to in Sanford, J. P., The Sanford Guide To Antimicrobial Therapy, 40th Edition (Antimicrobial Therapy, Inc. 2010). Any of the bacterial infections or disorders or symptoms thereof may or may not be recurring.

In an exemplary embodiment, the infection is a disease that is an AAD. Symptoms vary according to the degree of inflammation caused by the bacteria, ranging from slightly loose stools to bloody diarrhea, abdominal pain, and fever. The most severe cases may involve life-threatening dehydration, low blood pressure, toxic megacolon, and perforation of the large intestine. If a subject with, for example, a C. difficile infection develops diarrhea while taking antibiotics, the drugs are discontinued immediately unless they are essential. C. difficile-induced colitis without complications usually subsides on its own within 5 to 7 days after the antibiotic has been stopped. When it does, no other therapy is required.

In an exemplary embodiment, the disease is associated with C. difficile. For most cases of more severe C. difficile infection, the antibiotic metronidazole is usually effective against C. difficile, while the antibiotic vancomycin is reserved for the more severe cases. Symptoms return as a clinical recurrence in 20% to 30% of people with this disorder, and treatment with antibiotics is repeated. If diarrhea recurs repeatedly, prolonged antibiotic therapy may be needed. Rarely, Clostridium difficile-induced colitis is so severe that the person must be hospitalized to receive intravenous fluids, electrolytes (such as sodium, magnesium, calcium, and potassium), and blood transfusions. A temporary ileostomy (a surgically created connection between the small intestine and an opening in the abdominal wall that diverts stool from the large intestine and rectum) or surgical removal of the large intestine (colectomy) occasionally is needed in these severe cases as a lifesaving measure.

In an exemplary embodiment, the bacterium is C. difficile. C. difficile produces intestinal damage and diarrhea by releasing two exo-toxins, A and B, into the intestinal lumen. Toxin A, a 308-kDa heat-labile protein, elicits acute enteritis and secretion of fluid from ileum and colon of several animal species. The toxin elicits an inflammatory exudate containing lymphocytes, neutrophils, and serum proteins and pro-inflammatory cytokines that mediate a profound and rapid inflammatory response. The induction of fluid secretion and inflammation by toxin A involves extensive signaling cross-talk between epithelial cells, mast cells, sensory neurons, and inflammatory cells of the intestinal lamina propria.

The cellular mechanism of toxin A involves glucosylation of a threonine residue at position 37 on Rho, Rac, and cdc42, small GTP-binding proteins that regulate cell shape through modulation of the actin cytoskeleton. Monoglucosylation and inactivation of Rho proteins by the toxin causes severe cytoskeletal abnormalities in cultured and intact human colonocytes. However, the signal transduction pathways by which toxin A induces intestinal inflammation are not entirely known. Toxin A binds to a G protein-coupled receptor on the luminal aspect of the apical intestinal epithelial cell membrane and is then internalized where it activates MAPKs, intracellular calcium release, release of reactive oxygen species (ROS), and secretion of pro-inflammatory mediators. Toxin A releases prostaglandin E₂ (PGE₂) into the ileal lumen of intact rats and Alcantara reported that toxin A-induced water and electrolyte secretion in vivo was significantly blocked by a COX-2 inhibitor. COX-2 is induced by pro-inflammatory cytokines, lipopolysaccharide, growth factors, and infectious agents in a variety of cell types. PGE₂ is a potent stimulator of intestinal chloride and water secretion in mammalian gut, and PGE₂ is released during various forms of intestinal inflammation and infection.

Methods of treating or preventing a bacterial infection described herein comprise administering a pharmaceutically effective amount of the compound of Formula I and an anti-inflammatory agent to a subject. As used herein, the term “administer” and “administering” are used to mean introducing the compound of Formula I into a subject. When administration is for the purpose of treatment, the substance is provided at, or after the onset of, a symptom of a bacterial infection. The therapeutic administration of this substance serves to attenuate any symptom, or prevent additional symptoms from arising. When administration is for the purposes of preventing or reducing the likelihood a bacterial infection, the substance is provided in advance of any visible or detectable symptom, such as after the symptoms of the initial infection. The prophylactic administration of the substance serves to attenuate subsequently arising symptoms or prevent or reduce the likelihood of the symptoms from arising altogether. Accordingly, the compound of Formula I may be used for the prevention of one disease or disorder and concurrently treating another (e.g., prevention of AAC), while treating urinary AAD.

The route of administration of the compound of Formula I includes, but is not limited to, oral (such as an oral suspension), topical, transdermal, intranasal, vaginal, rectal, intraarterial, intramuscular, intraosseous, intraperitoneal, epidural and intrathecal. In an exemplary embodiment, the route of administration is oral.

Furthermore, the methods of treating or preventing a bacterial infection of the present invention also relate to co-administering one or more substances in addition to the compound of Formula I and an anti-inflammatory agent to the subject. The term “co-administer” indicates that each of at least two compounds are administered during a time frame wherein the respective periods of biological activity or effects overlap. Thus, the term includes sequential as well as coextensive administration of compounds. Similar to administering compounds, co-administration of more than one substance can be for therapeutic and/or prophylactic purposes. If more than one substance or compound is co-administered, the routes of administration of the two or more substances need not be the same. The scope of the invention is not limited by the identity of the substance which may be co-administered with the compound of Formula I. For example, compositions comprising the compound of Formula I may be co-administered with fluids or other substances that are capable of alleviating, attenuating, preventing or removing symptoms in a subject suffering from, exhibiting the symptoms of, or at risk of suffering from a bacterial infection. Types of fluid that can be co-administered with the compound of Formula I should be specific to the circumstances surrounding the particular subject that is suffering from, exhibiting the symptoms of, or at risk of suffering from a bacterial infection. For example, fluids that may be co-administered with the compound of Formula I include but are not limited to, electrolytes and/or water, salt solutions, such as sodium chloride and sodium bicarbonate, as well as whole blood, plasma, serum, serum albumin and colloid solutions.

As used herein and unless otherwise indicated, the phrase “therapeutically effective amount” (or “pharmaceutically effective amount”) of the compound of Formula I or a pharmaceutically acceptable salt or prodrug thereof is measured by the therapeutic effectiveness of a compound of the invention, wherein at least one adverse effect of a disorder is ameliorated or alleviated. In one embodiment, the term “therapeutically effective amount” means an amount of the compound of Formula I that is sufficient to provide the desired local or systemic effect and performance at a reasonable benefit/risk ratio attending any medical treatment. The response to the therapeutically effective amount may be a cellular, organ or tissue-specific response, or system or systemic response. In one embodiment, the phrase “therapeutically effective amount” of a composition of the invention is measured by the therapeutic effectiveness of a compound of the invention to alleviate at least one symptom associated with bacterial infections. Examples of therapeutically effective amounts include, but are not limited to those in the Examples section herein.

As used herein and unless otherwise indicated, the term “binders” refers to agents used to impart cohesive qualities to the powdered material. Binders, or “granulators” as they are sometimes known, impart cohesiveness to the tablet formulation, which insures the tablet remaining intact after compression, as well as improving the free-flowing qualities by the formulation of granules of desired hardness and size. Materials commonly used as binders include starch, gelatin, sugars, such as sucrose, glucose, dextrose, molasses, and lactose, natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, larch arabogalactan and the like.

As used herein and unless otherwise indicated, the term “carrier” refers to a diluent, adjuvant, excipient, or vehicle with which a composition is administered. Such pharmaceutical carriers can be sterile liquids, such as water and oils, including those of petroleum, animal, vegetable or synthetic origin, such as peanut oil, soybean oil, mineral oil, sesame oil and the like.

As used herein and unless otherwise indicated, the term “compound” means, collectively, a compound as described herein, and/or a pharmaceutically acceptable salt, solvate, hydrate, amorphous form and polymorph thereof. The compounds are identified herein by their chemical structures and/or chemical names. Where a compound is referred to by both a chemical structure and a chemical name, and that chemical structure and chemical name conflict, the chemical structure is determinative of the compound's identity. The compounds may contain one or more chiral centers and/or double bonds and may therefore exist as stereoisomers, such as double-bond isomers (i.e., geometric isomers), enantiomers, or diastereomers. According to the invention, the chemical structures depicted herein, and therefore the compounds, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures, and solvates and/or hydrates thereof. Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods.

In one embodiment, the pharmaceutical compositions used in the methods of the present invention comprise the compound of Formula I that is substantially stereomerically pure. In specific embodiments, the pharmaceutical compositions comprise the compound of Formula I that is at least about 75% pure, 80%, 85%, 90%, 91%, 92%, 93%, 94%, 95%, 96%, 97%, 98% or 99% pure, i.e., free from other stereoisomers, diastereoisomers, enantiomers, etc.

As used herein and unless otherwise indicated, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein and unless otherwise indicated, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

The term “MIC” or “minimum inhibitory concentration” refers to the lowest concentration of an antibiotic that is needed to inhibit growth of a bacterial isolate in vitro. A common method for determining the MIC of an antibiotic is to prepare several tubes containing serial dilutions of the antibiotic, that are then inoculated with the bacterial isolate of interest. The MIC of an antibiotic can be determined from the tube with the lowest concentration that shows no turbidity (no growth). The term “MIC₅₀” refers to the lowest concentration of antibiotic required to inhibit the growth of 50% of the bacterial strains tested within a given bacterial species. The term “MIC₉₀” refers to the lowest concentration of antibiotic required to inhibit the growth of 90% of the bacterial strains tested within a given bacterial species.

As used herein and unless otherwise indicated, the term “mixture of tiacumicins” refers to a composition containing at least one macrolide compound from the family of compounds known tiacumicins. In another embodiment, the term “mixture of tiacumicins” includes a mixture containing at least one member of the compounds known tiacumicins and the compound of Formula I, wherein the compound of Formula I is present in an amount of at least about 50%, 60%, 70%, 80%, 90%, 95%, 99%, 99.9%, or 99.99% by weight. In particular, the term “mixture of tiacumicins” refers to a compositions comprising the compound of Formula I, wherein the compound of Formula I has a relative retention time (“RTT”) ratio of 1.0, and further comprising at least one of compounds 101-112 in U.S. Pat. No. 7,378,508 which is incorporated by reference herein.

As used herein, and unless otherwise indicated, the terms “optically pure,” “stereomerically pure,” and “substantially stereomerically pure” are used interchangeably and mean one stereoisomer of a compound or a composition that comprises one stereoisomer of a compound and is substantially free of other stereoisomer(s) of that compound. For example, a stereomerically pure compound or composition of a compound having one chiral center will be substantially free of the opposite enantiomer of the compound. A stereomerically pure compound or composition of a compound having two chiral centers will be substantially free of other diastereomers of the compound. A typical stereomerically pure compound comprises at least about 80% by weight of one stereoisomer of the compound, i.e., free from other stereoisomers, diastereoisomers, enantiomers, etc., and about 20% or less by weight of other stereoisomers of the compound, more specifically at least about 90% by weight of one stereoisomer of the compound and about 10% or less by weight of the other stereoisomers of the compound, even more specifically, at least about 95% by weight of one stereoisomer of the compound and about 5% or less by weight of the other stereoisomers of the compound, and more specifically, at least about 97% by weight of one stereoisomer of the compound and about 3% or less by weight of the other stereoisomers of the compound.

As used herein and unless otherwise indicated, “pharmaceutically acceptable” refers to materials and compositions that are physiologically tolerable and do not typically produce an allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein and unless otherwise indicated, the term “pharmaceutically acceptable hydrate” means the compound of Formula I that further includes a stoichiometric or non-stoichiometric amount of water bound by non-covalent intermolecular forces.

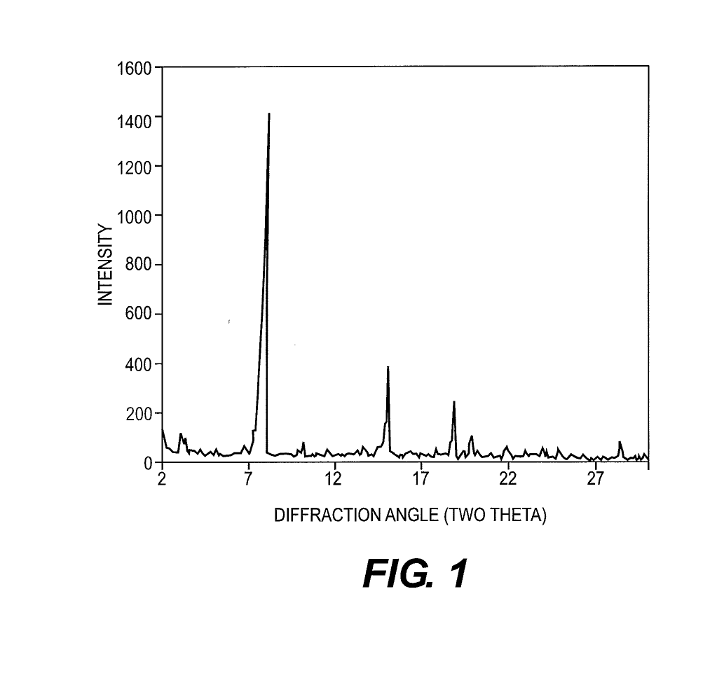

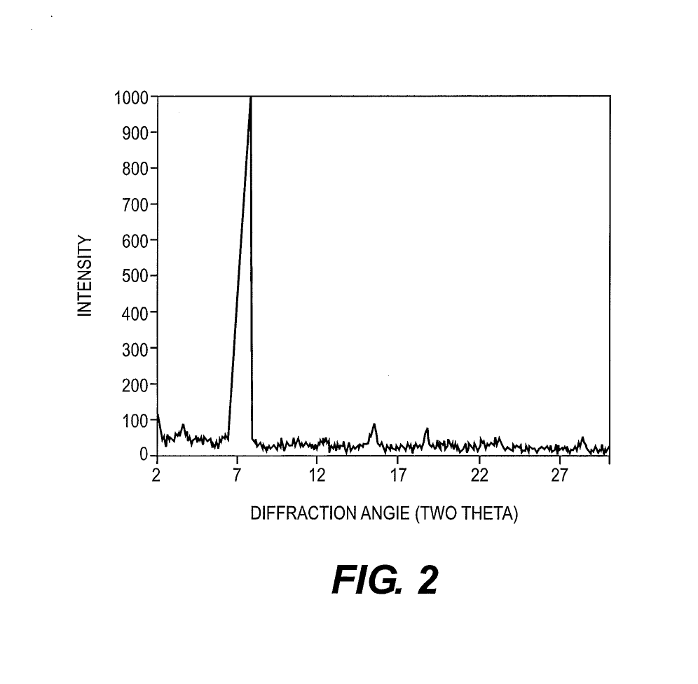

As used herein and unless otherwise indicated, the term “pharmaceutically acceptable polymorph” refers to the compound of Formula I that exists in several distinct forms (e.g., crystalline, amorphous), the invention encompasses all of these forms. In another embodiment, a pharmaceutically acceptable polymorph of a Compound of Formula I exhibits a representative powder diffraction pattern comprising at least peaks at the following diffraction angles 2θ of 7.7°, 15.0°, and 18.8°±0.04, or ±0.1, or ±0.15, or ±0.2, as shown in FIG. 1. In another embodiment, a pharmaceutically acceptable polymorph of a Compound of Formula I exhibits a representative powder diffraction pattern comprising at least peaks at the following diffraction angles 20 of 7.6°, 15.4°, and 18.8°±0.04, or ±0.1, or ±0.15, or ±0.2, as shown in FIG. 2.

Methods of preparing and characterizing select embodiments of pharmaceutically acceptable polymorphs are found in U.S. Pat. No. 7,378,508 incorporated herein by reference.

As used herein and unless otherwise indicated, the term “pharmaceutically acceptable prodrug” means a derivative of a modified polymorph of a compound of Formula I that can hydrolyze, oxidize, or otherwise react under biological conditions (in vitro or in vivo) to provide the compound of Formula I. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery (1995), and Design of Prodrugs (1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound of Formula I but can confer upon that compound advantageous properties in vivo, such as but not limited to uptake, duration of action, or onset of action, or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, amino acid amides, alkoxyacyl amides, and alkylaminoalkyl-carbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

The phrase “pharmaceutically acceptable salt(s),” as used herein includes but is not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds, included in the present compositions, which are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

In some embodiments, the methods of the invention encompass administering pharmaceutical compositions comprising a first polymorph of the compound of Formula I, a second polymorph of the compound of Formula I, other polymorphic forms, amorphous form or mixtures thereof of a mixture of tiacumicins with varying amounts of the compound of Formula I. Certain embodiments of the methods of the present invention may also comprise administering pharmaceutical compositions that are mixtures of tiacumicins for use in treating CDAD as well as AAD and AAC. In one specific embodiment, the mixture of tiacumicins contains from about 76% to about 100% of the compound of Formula I.

The present compositions, which comprise one or more crystalline polymorph or amorphous form of the compound of Formula I or the compound of Formula I within a mixture of tiacumicins may be administered by any convenient route, for example, peroral administration, parenteral administration, by infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a composition of the invention. In certain embodiments, more than one compound of Formula I and a mixture of tiacumicins are administered to a patient. Methods of administration include but are not limited to intradermal, intramuscular, intraperitoneal, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner, and will depend in part upon the site of the medical condition. In most instances, administration will result in the release of the crystalline polymorph or amorphous form of the compound of Formula I into the bloodstream.

In specific embodiments, it may be desirable to administer one or more crystalline polymorphs or amorphous forms of the compound of Formula I locally to the area in need of treatment. The administration may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the a crystalline polymorph or amorphous form of the compound of Formula I can be delivered in a vesicle, in particular a liposome (see Langer, 1990, Science 249:1527-1533; Treat, in Liposomes in The Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compositions of the invention can be delivered in a controlled release system. In one embodiment, a pump may be used (see Langer, supra; Sefton, 1987, CRC Crit. Ref Biomed. Eng. 14:201; Buchwald, 1980, Surgery 88:507; Saudek, 1989, N. Engl. J. Med. 321:574). In another embodiment, polymeric materials can be used. In yet another embodiment, a controlled-release system can be placed in proximity of the target of the compound of Formula I, e.g., the liver, thus requiring only a fraction of the systemic dose. Other controlled-release systems discussed in the review by Langer (1990) Science 249:1527-1533 may be used.

The present compositions will contain a therapeutically effective amount of a crystalline polymorphs or amorphous forms of the compound of Formula I, optionally more than one crystalline polymorph or amorphous form of the compound of Formula I, for example in purified form, together with a suitable amount of a pharmaceutically acceptable vehicle so as to provide the form for proper administration to the patient.

In a specific embodiment, the pharmaceutical composition contains one or more crystalline polymorphs or amorphous forms of the compound of Formula I. The crystalline polymorphs of the compound of Formula I include but not limited to the ones with the X-ray power diffraction pattern as shown in FIG. 1 and FIG. 2.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which the compound of Formula I is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention and pharmaceutically acceptable vehicles are preferably sterile. Water is an example of a vehicle of the compounds of the invention. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see U.S. Pat. No. 5,698,155). Other examples of suitable pharmaceutical vehicles are described in Remington's The Science and Practice of Pharmacy (2010). The pharmaceutical compositions may contain preserving agents, solubilising agents, stabilising agents, wetting agents, emulsifiers, sweeteners, colorants, odorants, salts, buffers, coating agents or antioxidants, such as but not limited to butylated hydroxytoluene (BHT). They may also contain therapeutically active agents in addition to the substance of the present invention.

In one embodiment, the compositions of the invention are administered orally. Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in a tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered crystalline polymorph or amorphous form of the compound of Formula I. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a crystalline polymorph or amorphous form of the compound of Formula I that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. Suitable dosage ranges for oral administration, however, are generally from about 0.001 milligram to 1000 milligrams of the compound of Formula I per kilogram body weight. In one embodiment, the oral dose is about 0.01 milligram to about 500 milligrams per kilogram body weight, or from about 0.1 milligram to about 100 milligrams per kilogram body weight, or from about 0.5 milligram to about 50 milligrams per kilogram body weight. In a specific embodiment, the oral dose is from about 1 milligram to about 10 milligrams per kilogram body weight. In a more specific embodiment, the oral dose is about 1 milligram of a crystalline polymorph or amorphous form of the compound of Formula I per kilogram body weight. The dosage amounts described herein refer to total amounts administered; that is, if more than one compound is administered, the preferred dosages correspond to the total amount of the compounds of the invention administered. The oral compositions described herein may contain from about 10% to about 95% active ingredient by weight, and the oral compositions may be dosed 1, 2, 3, 4, 5 or more times daily.

Suitable dosage ranges for intranasal administration are generally from about 0.01 pg/kg body weight to about 1 mg/kg body weight of the compound of Formula I. Suppositories generally contain from about 0.01 milligram to about 50 milligrams of the compound of Formula I per kilogram body weight and comprise active ingredient in the range of from about 0.5% to about 10% by weight. Recommended dosages for intradermal, intramuscular, intraperitoneal, epidural, sublingual, intracerebral, intravaginal, transdermal administration or administration by inhalation are in the range of from about 0.001 milligram to about 1000 milligrams per kilogram of body weight of the compound of Formula I. Suitable doses of the compounds of the invention for topical administration are in the range of from about 0.001 milligram to about 1 milligram of the compound of Formula I, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more crystalline polymorph or amorphous form of the compound of Formula I. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one crystalline polymorphs or amorphous forms of the compound of Formula I.

Methods of manufacturing the compound of Formula I, including select polymorphs thereof are disclosed in U.S. Pat. No. 7,378,508, which is incorporated by reference.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

The following examples describe the combination of fidaxomicin with various anti-inflammatory compounds in a mouse Clostridium difficile infection (CDI) model.

### EXAMPLES

### Example 1

### Mouse Model of CDI

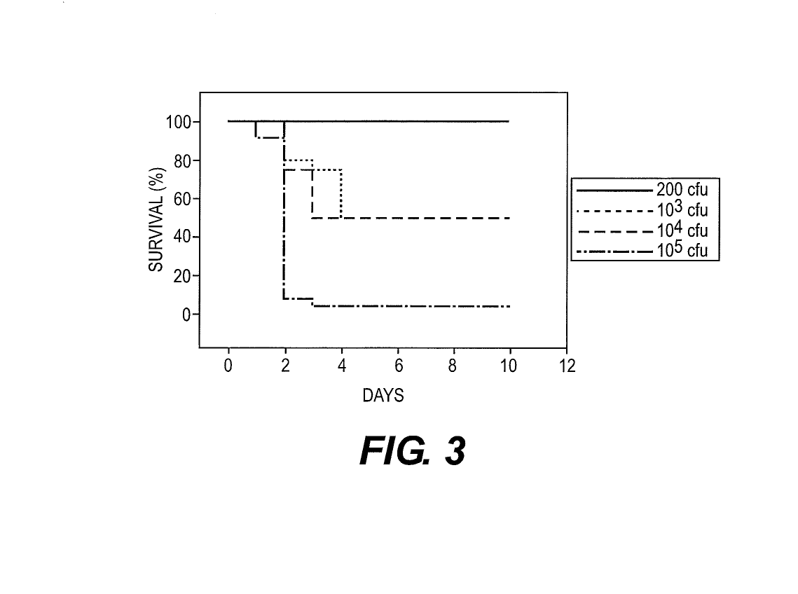

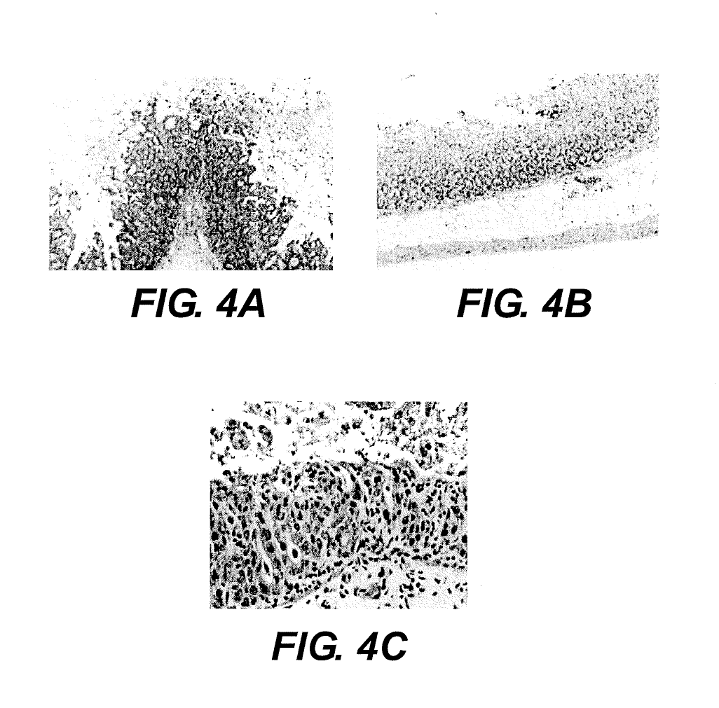

Although hamsters have been used to investigate disease pathogenesis and treatment in CDI, they are not ideal models because of the lack of hamster specific reagents and, more importantly, the low similarity to the human CDI disease. Upon infection, hamsters develop fulminant and rapidly fatal CDI unlike humans that exhibit a spectrum of disease severity and outcomes. These limitations of the hamster CDI model thus hamper its utility to study clinical relevant therapies. Accordingly, the inventors have used an established mouse model of antibiotic-induced CDI that resembles more closely the disease in human (Gastroenterology 2008, 135: 1984-1992). In this model, C57BL/6 mice were exposed to a mixture of oral antibiotics (kanamycin, gentamicin, colistin, metronidazole, and vancomycin) for 3 days followed by a parenteral administration of clindamycin two days later, and then challenged with different doses of C. difficile 1 day later. CDI caused diarrhea and weight loss in mice exposed to antibiotic and C. difficile with disease severity varying from fulminant to minimal in proportion to the challenge dose as shown in FIG. 3. Typical CDI histologic features are shown in FIGS. 4A-4C (Gastroenterology 2008, 135: 1984-1992).

FIG. 4A shows enteritis associated with proliferative ulcerations, epithelial necrosis, and release of inflammatory exudates and necrotic cellular material into the intestinal lumen (upper right), consistent with pseudomembranous colitis (PMC). FIG. 4B shows marked submucosal edema without submucosal inflammation (pale area in lower third of frame), as seen in severe PMC in humans. FIG. 4C shows high power view of mucosal proliferation, epithelial necrosis, and recruitment of inflammatory cells, (predominantly neutrophils).

In summary, a mouse model of CDI is presented which resembles the disease in human in relation to the predisposing factors, target organ, and disease manifestation which is demonstrated in the involvement of the entire colon, pseudomembrane formation, variation of the disease severity, and the use of antibiotics (Gastroenterology 2008, 135: 1984-1992).

### Example 2

### Evaluation of Test Compounds in Mouse Model

C57BL/6 mice will be housed in cages with free access to food and water. Animals will be treated with a mixture of oral antibiotics (kanamycin, gentamicin, colistin, metronidazole and vancomycin) for 3 days (Gastroenterology 2008, 135(6):1984-1992) followed by a parenteral administration of clindamycin phosphate (10 mg/kg s.c.) two days later [Day 0]. Mice will be challenged by gavage with 10² to 10² cfu of toxinogenic C. difficile one day later [Day 1]. A mild moderate to fulminant colitis is expected to develop 1 to 5 days after the administration of C. difficile. If left untreated, C. difficile infection can progress to severe colitis and death in the majority of animals when challenged with a high dose of the bacteria.

For these experiments, mice will be treated with C. difficile bacteria (˜10⁶ cfu of C. difficile strain 10465) to cause moderate to severe colitis. Some animals will be treated with fidaxomicin (1 to 50 mg/kg). The test compounds will consist of mesalamine and/or budesonide. These will be administered to groups of animals either alone or in conjunction with fidaxomicin. Dosing (by gavage) will commence 1-2 days after C. difficile challenge at the onset of clinically evident CU and will continue daily for 5 days.

TABLE 1
Example of experimental group and dosing schedule

| | | | | | Days of |
|---|---|---|---|---|---|
| No. | n = | Treatment ^{a, b} | Dose | Route | dosing ^{c} |
| 1 | 8 to 10 | Positive control | Vehicle | po (gavage) | 3, 4, 5, |
| | | | | | 6 and 7 |
| 2 | 8 to 10 | Fidaxomicin alone | TBD | po (gavage) | 3, 4, 5, |
| | | | | | 6 and 7 |
| 3 | 8 to 10 | Test compound A | TBD | po (gavage) | 3, 4, 5, |
| | | | | | 6 and 7 |
| 4 | 8 to 10 | Test compound B | TBD | po (gavage) | 3, 4, 5, |
| | | | | | 6 and 7 |
| 5 | 8 to 10 | Fidaxomicin plus | TBD ^{c} | po (gavage) | 3, 4, 5, |
| | | test compound A | | | 6 and 7 |
| 6 | 8 to 10 | Fidaxomicin plus | TBD ^{c} | po (gavage) | 3, 4, 5, |
| | | test compound B | | | 6 and 7 |
| 7 | 8 to 10 | Negative control | Vehicle | po (gavage) | 3, 4, 5, |
| | | | | | 6 and 7 |

^{a} On Day 0, all except negative control animals receive clindamycin (10 mg/kg s.c.)
^{b} On Day 1, all except negative control animals receive gavage with ~10⁶ cfu pf C. difficile
^{c} Lower or higher doses of fidaxomicin, mesalamine and/or budesonide can be tested

The animals will be weighed and monitored daily for morbidity and presence of diarrhea. Animals judged to be in a moribund state will be euthanized prior to the end of dosing period.

The following study parameters will be measured: (1) Survival, (2) Weight loss/gain, (3) Severity of colitis as determined by (a) by histopathology with semi-quantitative scoring, (b) by measurement of inflammatory cytokines e.g. TNFα, CXC chemokine (KC), (c) by measurement of PMN infiltration using MPO. Data will be compared to determine the severity of CM disease and associated inflammation across study groups.

Although the present invention has been described in detail with reference to examples above, it is understood that various modifications can be made without departing from the spirit of the invention. Accordingly, the invention is limited only by the following claims.

1. A method of treating an infection in a mammal, wherein the infection is caused by the presence of a bacterium, comprising administering to a mammal in need thereof an effective amount of a combination of a compound of Formula I 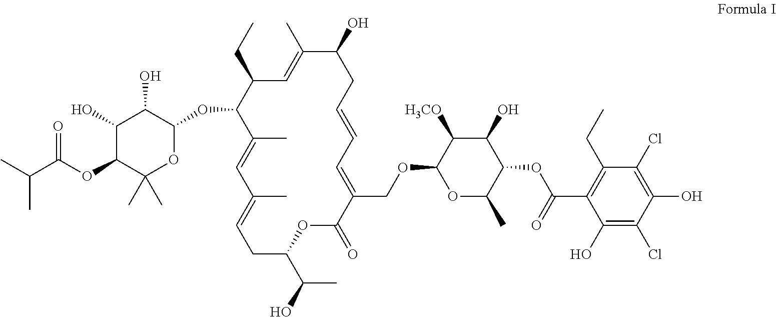 [SMILES: CCc1c(Cl)c(O)c(Cl)c(O)c1C(=O)O[C@H]1[C@H](O)[C@H](OC)[C@H](OC/C2=C\C=C\C[C@H](O)/C(C)=C/[C@H](CC)[C@@H](O[C@@H]3OC(C)(C)[C@@H](OC(=O)C(C)C)[C@H](O)[C@@H]3O)/C(C)=C/C(C)=C/C[C@@H]([C@@H](C)O)OC2=O)O[C@@H]1C]
and an anti-inflammatory agent.

2. The method of claim 1, wherein the compound of Formula I is administered simultaneously with the anti-inflammatory agent.

3. The method of claim 1, wherein the compound of Formula I is administered at a time that is different from when the anti-inflammatory agent is administered.

4. The method of claim 2, wherein the compound of Formula I and the anti-inflammatory agent are administered in a solid dosage formulation.

5. The method of claim 4, wherein the anti-inflammatory agent is present in the solid formulation in an amount of about 2% to about 80% by weight.

6. The method of claim 4, wherein the anti-inflammatory agent is present in the solid formulation in an amount of about 2% to about 5% by weight.

7. The method of claim 1, wherein the bacterium is a gram-positive bacterium.

8. The method of claim 1, wherein the bacterium is a gram-negative bacterium.

9. The method of claim 1, wherein the bacterium is selected from the group consisting of Clostridium spp., Staphylococcus spp., Enterococcus spp. and combinations thereof.

10. The method of claim 1, wherein the bacterium is Clostridium spp.

11. The method of claim 1, wherein the bacterium is selected from the group consisting of C. difficile, C. perfringens, S. aureus and combinations thereof.

12. The method of claim 1, wherein the bacterium is C. difficile.

13. The method of claim 1, wherein the infection is selected from the group consisting of diarrhea and colitis.

14. The method of claim 1, wherein the infection is infectious diarrhea.

15. The method of claim 1, wherein the infection is colitis.

16. The method of claim 1, wherein the infection is C. difficile-associated diarrhea.

17. The method of claim 9, wherein the Staphylococcus spp. is methicillin-resistant.

18. The method of claim 9, wherein the Staphylococcus spp. is methicillin-sensitive.

19. The method of claim 17, wherein the Staphylococcus spp. is Staphylococcus aureus.

20. The method of claim 9, wherein the Enterococcus spp. is vancomycin-resistant.

21. The method of claim 9, wherein the Enterococcus spp. is vancomycin-sensitive.

22. The method of claim 1, wherein the compound of Formula I and the anti-inflammatory agent are administered as a pharmaceutical composition comprising a pharmaceutically acceptable excipient.

23. The method of claim 1, wherein the compound of Formula I contains at least 93% of the R-stereoisomer.

24. The method of claim 1, wherein the mammal is a human.

25. The method of claim 1, wherein the combination of the compound of Formula I and the anti-inflammatory agent is administered in an amount of about 50 mg to about 1,000 mg one to three times daily within three to fifteen days.

26. The method of claim 1, wherein the combination of the compound of Formula I and the anti-inflammatory agent is administered in an amount of about 100 mg to about 400 mg once or twice daily.

27. The method of claim 1, wherein the combination of the compound of Formula I and the anti-inflammatory agent is administered in an amount of about 200 mg once daily.

28. The method of claim 1, wherein the anti-inflammatory agent is selected from the group consisting of mesalamine, sulfasalazine, balsalazide, inflixamab, olsalazine, and budesonide.

29. The method of claim 1, wherein the anti-inflammatory agent is mesalamine.

30. The method of claim 1, wherein the anti-inflammatory agent is budesonide.

31. The method of claim 1, wherein the anti-inflammatory agent is a 5-aminosalicylic acid compound.

32. A pharmaceutical composition comprising an effective amount of a combination of a compound of Formula I 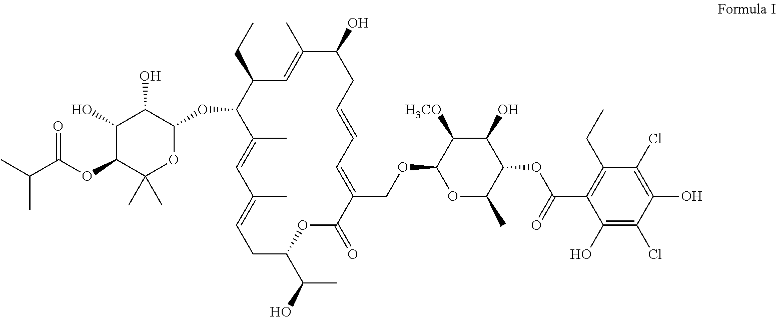 [SMILES: CCc1c(Cl)c(O)c(Cl)c(O)c1C(=O)O[C@H]1[C@H](O)[C@H](OC)[C@H](OC/C2=C\C=C\C[C@H](O)/C(C)=C/[C@H](CC)[C@@H](O[C@@H]3OC(C)(C)[C@@H](OC(=O)C(C)C)[C@H](O)[C@@H]3O)/C(C)=C/C(C)=C/C[C@@H]([C@@H](C)O)OC2=O)O[C@@H]1C]
and an anti-inflammatory agent.

33. The composition of claim 32, wherein the anti-inflammatory agent is selected from the group consisting of mesalamine, sulfasalazine, balsalazide, inflixamab, olsalazine, and budesonide.

34. The composition of claim 32, wherein the anti-inflammatory agent is mesalamine.

35. The composition of claim 32, wherein the anti-inflammatory agent is budesonide.

36. The composition of claim 32, wherein the anti-inflammatory agent is a 5-aminosalicylic acid compound.
